(19) United States
(12) Patent Application Publication
(10) Pub. No.: US 2025/0179445 A1
(43) Pub. Date: Jun. 5, 2025

(54) RECOMBINANT KOD POLYMERASE

(71) Applicant: BGI SHENZHEN, Shenzhen (CN)

(72) Inventors: Lili ZHAI, Shenzhen (CN); Lin WANG, Shenzhen (CN); Wenwei ZHANG, Shenzhen (CN); Yuliang DONG, Shenzhen (CN); Yue ZHENG, Shenzhen (CN); Fen LIU, Shenzhen (CN)

(21) Appl. No.: 18/958,402

(22) Filed: Nov. 25, 2024

Related U.S. Application Data
(63) Continuation-in-part of application No. 17/270,489, filed on Feb. 23, 2021, now Pat. No. 12,168,785.

(51) Int. Cl. C12N 9/12; C12N 15/70; C12Q 1/6844

(52) U.S. Cl. CPC C12N 9/1252; C12N 15/70; C12Q 1/6844; C12Y 207/07007

(57) ABSTRACT

Provided is a recombinant KOD polymerase, which is the following A) or B): the polymerase shown in A) is a protein having DNA polymerase activity that is obtained by modifying amino acid residues in at least one of the following 18 positions in a wild-type KOD DNA polymerase amino acid sequence: 675th, 385th, 710th, 674th, 735th, 736th, 606th, 709th, 347th, 349th, 590th, 676th, 389th, 589th, 680th, 384th, 496th and 383rd; the polymerase described by B) is a protein having DNA polymerase activity that is derived from A) by adding a tag sequence to an end of the amino acid sequence of the protein shown in A).

### CROSS-REFERENCE TO RELATED APPLICATIONS

This application is a Divisional application of U.S. patent application Ser. No. 17/270,489, filed Feb. 23, 2021, which is a National Stage Entry of PCT/CN2018/103764, filed Sep. 3, 2018, each of which applications is hereby incorporated herein by reference in its entirety.

### INCORPORATION BY REFERENCE STATEMENT

This statement, made under Rules 77(b)(5)(ii) and any other applicable rule incorporates into the present specification of an XML file for a “Sequence Listing XML” (see Rule 831(a)), submitted via the USPTO patent electronic filing system or on one or more read-only optical discs (see Rule 1.52(e)(8)), identifying the names of each file, the date of creation of each file, and the size of each file in bytes as follows:
File name: 218_013USA_sequence
Creation date: Nov. 20, 2023
Byte size: 11,536

### TECHNICAL FIELD

The present disclosure relates to the field of biotechnology, specifically to a recombinant KOD polymerase.

### INTRODUCTION

The role of DNA polymerase is to replicate DNA quickly and accurately, which plays an important role in transmitting genetic information precisely and maintaining the stability of genetic material in living organism. There are five types of DNA polymerases in Escherichia coli, and according to the sequence homology of these DNA polymerases, they are divided into A, B, C and other types. The total folding structure of DNA polymerase is similar with that of human hand, which is divided into three parts: palm, thumb and finger. Although the structures of the fingers and palm domains of different types of DNA polymerases vary greatly, the palm domains for catalysis differ slightly. The major amino acid sequence of the active sites of DNA polymerase are conserved, but these conserved active sites are also relatively easy to change, and unnatural specific dNTPs can be incorporated to keep the activity of DNA polymerase from decreasing significantly (see U.S. Pat. No. 6,602,695 of Patel et al.).

KOD DNA polymerase belongs to B-family DNA polymerase, which can replicate DNA quickly and accurately. It is a heat-resistant DNA polymerase with a wide range of applications. One of the most important applications is in genome sequencing, such as SBS sequencing, which use nucleotides modified in the 3′ sugar hydroxyl group to block the addition of other nucleotides. The use of nucleotides with 3′ blocking groups allows the incorporation of nucleotides into the polynucleotide chain in a controlled manner. After the addition of each nucleotide, the presence of the 3′ blocking group prevents other nucleotides from being incorporated into the chain. After removing the blocking group, the natural free 3′ hydroxyl group is restored for adding the next nucleotide. However, there are many technical defects with current SBS sequencing, such as short read length of sequencing and slow reaction rate. KOD polymerase is an enzyme product specifically improved to address these technical defects by removing the 3′-5′ exonuclease activity of wild-type KOD polymerase. A certain amount of optimized mutation sites were screened by computer simulation and prediction of the DNA binding region, dNTP catalytic site and other protein domains. Experimental screening of the mutated KOD polymerases yielded a batch of usable mutation site information, and it was found that the KOD polymerase can be modified by incorporating some unnatural amino acids. At present this type of products have been used in sequencing (see patent number of Liu Fen et al.).

### DISCLOSURE

In one aspect, provided is a protein, which is A) or B) as follows:
A) the protein shown is a protein that has DNA polymerase activity obtained by modifying amino acid residues in at least one of the 18 positions 675, 385, 710, 674, 735, 736, 606, 709, 347, 349, 590, 676, 389, 589, 680, 384, 496 and 383 of the amino acid sequence of wild-type KOD DNA polymerase;
B) the protein shown is a protein derived from A) that has DNA polymerase activity by adding a tag sequence to the end of the amino acid sequence of the protein shown in A).

In one embodiment, wherein:
A) the protein shown is a protein that has DNA polymerase activity obtained by modifying amino acid residues in at least two of the following 18 positions 675, 385, 710, 674, 735, 736, 606, 709, 347, 349, 590, 676, 389, 589, 680, 384, 496 and 383 of the amino acid sequence of wild-type KOD DNA polymerase.

In one embodiment, A) the protein shown is a protein that has DNA polymerase activity obtained by modifying amino acid residues in at least three of the following 18 positions 675, 385, 710, 674, 735, 736, 606, 709, 347, 349, 590, 676, 389, 589, 680, 384, 496 and 383 of the amino acid sequence of wild-type KOD DNA polymerase.

In another embodiment, A) the protein shown is a protein that has DNA polymerase activity obtained by modifying amino acid residues in at least four of the following 18 positions 675, 385, 710, 674, 735, 736, 606, 709, 347, 349, 590, 676, 389, 589, 680, 384, 496 and 383 of the amino acid sequence of wild-type KOD DNA polymerase.

In another embodiment, A) the protein shown is a protein that has DNA polymerase activity obtained by modifying amino acid residues in at least five of the following 18 positions 675, 385, 710, 674, 735, 736, 606, 709, 347, 349, 590, 676, 389, 589, 680, 384, 496 and 383 of the amino acid sequence of wild-type KOD DNA polymerase.

In another embodiment, A) the protein shown is a protein that has DNA polymerase activity obtained by modifying amino acid residues in at least six of the following 18 positions 675, 385, 710, 674, 735, 736, 606, 709, 347, 349, 590, 676, 389, 589, 680, 384, 496 and 383 of the amino acid sequence of wild-type KOD DNA polymerase.

In another embodiment, the modification is amino acid substitution.

In another embodiment, the amino acid substitution is at least one of the following:
the alanine at position 675 is substituted with phenylalanine or methionine;
the glutamate at position 385 is substituted with methionine or tryptophan;
the isoleucine at position 710 is substituted with serine or histidine;
the lysine at position 674 is substituted with leucine or cysteine; the asparagine at position 735 is substituted with histidine;
the glutamine at position 736 is substituted with glutamate or alanine;
the arginine at position 606 is substituted with histidine or methionine;
the arginine at position 709 is substituted with serine or histidine;
the serine at position 347 is substituted with isoleucine or methionine;
the threonine at position 349 is substituted with phenylalanine or isoleucine;
the threonine at position 590 is substituted with lysine or leucine;
the threonine at position 676 is substituted with lysine or tyrosine;
the valine at position 389 is substituted with isoleucine or methionine;
the valine at position 589 is substituted with histidine or glutamine;
the valine at position 680 is substituted with methionine or glutamate;
the tyrosine at position 384 is substituted with phenylalanine or tryptophan;
the tyrosine at position 496 is substituted with leucine or isoleucine;
the serine at position 383 is substituted with threonine.

In another embodiment, the amino acid sequence of wild-type KOD DNA polymerase is shown in SEQ ID NO: 1.

In another embodiment, provided is a DNA molecule encoding any of the disclosed proteins.

In another embodiment, provided is an expression cassette, recombinant vector, recombinant bacteria or transgenic cell line comprising the DNA molecule.

In another embodiment, provided is a use of any of the proteins as a DNA polymerase; or a use of the DNA molecule or the expression cassette, recombinant vector, recombinant bacteria or transgenic cell line in the preparation of DNA polymerase.

In another embodiment, provided is a use of any of the proteins, the DNA molecule or the expression cassette, recombinant vector, recombinant bacteria or transgenic cell in sequencing;
or a use of any of the proteins, the DNA molecule or the expression cassette, recombinant vector, recombinant bacteria or transgenic cell in the preparation of products for sequencing.

In another aspect, provided is a protein, wherein the sequence of the protein has at least 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% identity with the wild-type KOD DNA polymerase, and has the following substitutions in the corresponding wild-type KOD DNA polymerase amino acid sequence:
the threonine at position 676 with lysine; the valine at position 589 with histidine; and the valine at position 680 with methionine;
wherein the protein has DNA polymerase activity;
wherein the amino acid sequence of wild-type KOD DNA polymerase is shown in SEQ ID NO: 1.

In one embodiment, the sequence of the protein has substitutions at least at one of the following positions in the corresponding wild-type KOD DNA polymerase amino acid sequence: 383, 384, 389, 349, 496, and/or 709, and the protein has DNA polymerase activity.

In one embodiment, the sequence of the protein comprises the tag sequence in the terminal of the protein, and the protein has DNA polymerase activity.

In one embodiment, the sequence of the protein comprises the tag sequence in the terminal of the protein, and the protein has DNA polymerase activity.

In one embodiment, the sequence of the protein has at least one of the following substitutions in the corresponding wild-type KOD DNA polymerase amino acid sequence:
the serine at position 383 with threonine;
the tyrosine at position 384 with phenylalanine or tryptophan;
the valine at position 389 with isoleucine or methionine;
the threonine at position 349 with phenylalanine or isoleucine;
the tyrosine at position 496 with leucine or isoleucine;
the arginine at position 709 with serine or histidine.

In one embodiment, the sequence of the protein has the following substitutions in the corresponding wild-type KOD DNA polymerase amino acid sequence: the threonine at position 676 with lysine, the valine at position 589 with histidine, the valine at position 680 with methionine, and the tyrosine at position 384 with phenylalanine; and the protein has DNA polymerase activity.

In one embodiment, the sequence of the protein has the following substitutions in the corresponding wild-type KOD DNA polymerase amino acid sequence: the threonine at position 676 with lysine, the valine at position 589 with histidine, the valine at position 680 with methionine, the threonine at position 349 with phenylalanine, and the tyrosine at position 496 with leucine;
and the protein has DNA polymerase activity.

In one embodiment, the sequence of the protein has the following substitutions in the corresponding wild-type KOD DNA polymerase amino acid sequence: the threonine at position 676 with lysine, the valine at position 589 with histidine, the valine at position 680 with methionine, the serine at position 383 with threonine, and the tyrosine at position 384 with phenylalanine;
and the protein has DNA polymerase activity.

In one embodiment, the sequence of the protein has the following substitutions in the corresponding wild-type KOD DNA polymerase amino acid sequence: the threonine at position 676 with lysine, the valine at position 589 with histidine, the valine at position 680 with methionine, the serine at position 383 with threonine, the tyrosine at position 384 with phenylalanine, and the arginine at position 709 with serine; and the protein has DNA polymerase activity.

In one embodiment, the sequence of the protein has the following substitutions in the corresponding wild-type KOD DNA polymerase amino acid sequence: the threonine at position 676 with lysine, the valine at position 589 with histidine, the valine at position 680 with methionine, the serine at position 383 with threonine, the tyrosine at position 384 with phenylalanine, and the valine at position 389 with isoleucine;
and the protein has DNA polymerase activity.

In one embodiment, a DNA molecule encodes the protein.

In another embodiment, a recombinant expression vector comprises the DNA molecule.

In another embodiment, provided is a transformant, wherein the transformant is obtained by transducting the recombinant expression vector into host cell;
preferably the host cell is bacteria cell;
more preferably, the bacteria cell is BL21.

In another embodiment, provided is a method for preparing a DNA polymerase, comprising culturing the transformant to express the DNA polymerase;
preferably the method further comprising purifying the DNA polymerase.

In another embodiment, provided is a kit, wherein the kit comprises the protein; preferably the kit further comprising the specification.

In another embodiment, provided is a method for amplifying DNA, wherein the method comprising using the protein.

In another embodiment, provided is a method for amplifying DNA, wherein the method comprising using the kit.

A purpose of the present application is to provide a recombinant KOD polymerase.

The recombinant KOD polymerase provided by the present invention is a protein which is A) or B) as follows:
A) the protein shown is a protein that has DNA polymerase activity obtained by modifying amino acid residues in at least one of the 18 positions 675, 385, 710, 674, 735, 736, 606, 709, 347, 349, 590, 676, 389, 589, 680, 384, 496 and 383 of the amino acid sequence of wild-type KOD DNA polymerase;
B) the protein shown is a protein derived from A) that has DNA polymerase activity by adding a tag sequence to the end of the amino acid sequence of the protein shown in A).

In the protein as defined above, A) the protein shown is a protein that has DNA polymerase activity obtained by modifying amino acid residues in at least two of the following 18 positions675, 385, 710, 674, 735, 736, 606, 709, 347, 349, 590, 676, 389, 589, 680, 384, 496 and 383 of the amino acid sequence of wild-type KOD DNA polymerase.

In the protein as defined above, A) the protein shown is a protein that has DNA polymerase activity obtained by modifying amino acid residues in at least three of the following 18 positions 675, 385, 710, 674, 735, 736, 606, 709, 347, 349, 590, 676, 389, 589, 680, 384, 496 and 383 of the amino acid sequence of wild-type KOD DNA polymerase.

In the protein as defined above, A) the protein shown is a protein that has DNA polymerase activity obtained by modifying amino acid residues in at least four of the following 18 positions 675, 385, 710, 674, 735, 736, 606, 709, 347, 349, 590, 676, 389, 589, 680, 384, 496 and 383 of the amino acid sequence of wild-type KOD DNA polymerase.

In the protein as defined above, A) the protein shown is a protein that has DNA polymerase activity obtained by modifying amino acid residues in at least five of the following 18 positions 675, 385, 710, 674, 735, 736, 606, 709, 347, 349, 590, 676, 389, 589, 680, 384, 496 and 383 of the amino acid sequence of wild-type KOD DNA polymerase.

In the protein as defined above, A) the protein shown is a protein that has DNA polymerase activity obtained by modifying amino acid residues in at least six of the following 18 positions 675, 385, 710, 674, 735, 736, 606, 709, 347, 349, 590, 676, 389, 589, 680, 384, 496 and 383 of the amino acid sequence of wild-type KOD DNA polymerase.

In the protein as defined above, the modification is amino acid substitution.

In the protein as defined above, the amino acid substitution is at least one of the following:
the alanine at position 675 is substituted with phenylalanine or methionine;
the glutamate at position 385 is substituted with methionine or tryptophan;
the isoleucine at position 710 is substituted with serine or histidine;
the lysine at position 674 is substituted with leucine or cysteine;
the asparagine at position 735 is substituted with histidine;
the glutamine at position 736 is substituted with glutamate or alanine;
the arginine at position 606 is substituted with histidine or methionine;
the arginine at position 709 is substituted with serine or histidine;
the serine at position 347 is substituted with isoleucine or methionine;
the threonine at position 349 is substituted with phenylalanine or isoleucine;
the threonine at position 590 is substituted with lysine or leucine;
the threonine at position 676 is substituted with lysine or tyrosine;
the valine at position 389 is substituted with isoleucine or methionine;
the valine at position 589 is substituted with histidine or glutamine;
the valine at position 680 is substituted with methionine or glutamate;
the tyrosine at position 384 is substituted with phenylalanine or tryptophan;
the tyrosine at position 496 is substituted with leucine or isoleucine;
the serine at position 383 is substituted with threonine.

In the protein as defined above, the amino acid sequence of wild-type KOD DNA polymerase is shown in SEQ ID NO: 1.

The protein shown in A) is a point mutant of KOD DNA polymerase, specifically a single point mutant of Table 1 in the examples or a multiple-point mutant of Table 2 in the examples.

A DNA molecule encoding the protein as defined above also falls within the scope of the present invention.

An expression cassette, recombinant vector, recombinant bacteria or transgenic cell line comprising the DNA molecule as defined above also falls within the scope of the present invention.

A use of the protein as defined above as a DNA polymerase also falls within the scope of the present invention.

Or the DNA molecule or the expression cassette, recombinant vector, recombinant bacteria or transgenic cell line as define above in the preparation of DNA polymerase falls within the scope of the present invention.

Ause of the protein, the DNA molecule or the expression cassette, recombinant vector, recombinant bacteria or transgenic cell line as defined above in sequencing falls within the scope of the present invention; the sequencing as defined above is specifically DNA sequencing.

Or a use of the protein, the DNA molecule or the expression cassette, recombinant vector, recombinant bacteria or transgenic cell line as defined above in the preparation of products for sequencing in the preparation of products for sequencing.

In the use defined above, the product is a kit.

Based on the kinetic simulation and statistical inference of the KOD polymerase, the present invention obtains mutation sites that can be used for experimental screening. And experimental program was designed with semi-rational design, enzyme variant library construction and high-throughput screening to improve the catalytic, physical and chemical properties of polymerase and develop DNA polymerases suitable for DNB sequencing attached to the chip surface. Finally, the recombinant DNA polymerases were isolated and purified.

### DESCRIPTION OF THE DRAWINGS

FIG. 1 shows a schematic diagram of the structure of recombinant KOD DNA polymerase.

FIG. 2 shows the measurement of the purity of wild-type KOD DNA polymerase fusion protein.

FIG. 3 shows the Michaelis-Menten curve of wild-type and recombinant KOD DNA polymerase fusion protein.

### THE MOST PREFERABLE EMBODIMENT TO IMPLEMENT THE INVENTION

The experimental methods used in the following examples are conventional methods unless otherwise specified.

The materials and reagents used in the following examples are commercially available unless otherwise specified.

### Example 1

### Preparation of KOD DNA Polymerase Mutant

In the present invention, the expression vectors comprising KOD DNA polymerase or mutants thereof was constructed using DNA 2.0 Electra™ Cloning Reagents Kit, and His tag was used for Ni column affinity purification.

### 1. Preparation of Wild-Type KOD DNA Polymerase

The amino acid sequence of wild-type KOD DNA polymerase is SEQ ID NO: 1 in Sequence Listing, and its coding gene sequence is SEQ ID NO: 2 in Sequence Listing.

(1) Construction of Wild-Type Expression Vector pD441-WT

The wild-type recombinant expression vector pD441-WT was obtained by recombining the gene encoding wild-type KOD DNA polymerase fused with His tag to vector pD441-pelB (DNA2.0, pD441-pelB) according to the operation steps of the instruction of Electra™ Cloning Reagents Kit (DNA2.0, pD441-pelB). The gene encoding wild-type KOD DNA polymerase fused with His tag was expressed using the signal peptide on vector pD441-pelB.

The nucleotide sequence of gene encoding wild-type KOD DNA polymerase fused with His tag was a sequence obtained by linking 6×His tag codons at the 3′ end of SEQ ID NO: 2.

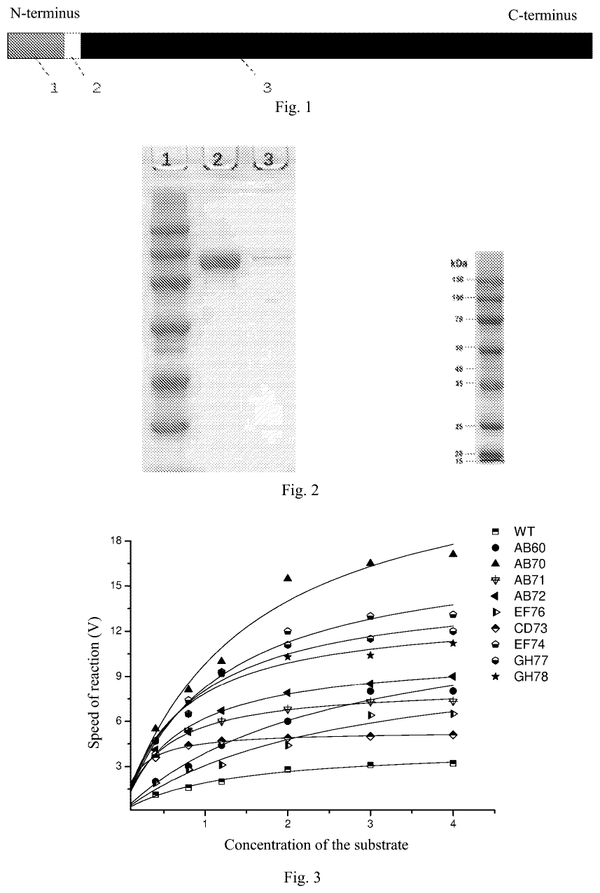

The amino acid sequence of wild-type KOD DNA polymerase fusion protein was obtained by connecting 6×His tags with the C-terminus of the amino acid of SEQ ID NO: 1, as shown in FIG. 1, wherein 1 represents the signal peptide pelB; 2 represents the purification tag His of fusion protein; 3 represents Wild-type KOD DNA polymerase.

### (2) Construction of Recombinant Bacteria

The wild-type recombinant expression vector pD441-WT was transducted into E. coli BL21 competent cells (purchased from TransGen Biotech Co., Ltd.), and coated on a plate containing 50 μg/ml of kanamycin to select positive colonies. 3-5 positive colonies were selected and then identified with PCR using primers Cloning-F (SEQ ID NO: 3 in the Sequence Listing) and Cloning-R (SEQ ID NO: 4 in the Sequence Listing). A fragment with a size of 2800 bp, which is basically consistent with the predicted theoretical value, was obtained and determined as a positive clone by comparison of the sequencing results and named BL21/pD441-WT.

SEQ ID NO: 3: Cloning-F:
5′GGTTTTTTTATGGGGGGAGTTTAGG 3′
SEQ ID NO: 4: Cloning-R:
5′CATCTCATCTGTAACATCATTGGCA 3′

### (3) Expression and Purification of Wild-Type KOD DNA Polymerase Fusion Protein

A single colony of BL21/pD441-WT was inoculated and cultured overnight in 50ml LB liquid medium (containing Kan50 μg/ml) at 37° C. and 220 rpm/min. Then the cultured bacteria solution was diluted 1:100 the next day and transferred to IL LB liquid medium (containing 50 μg/ml of Kan), then cultured in a shaker at 37° C. and 220 rpm/min to OD 600 of 0.5-0.8. Next, a 0.5 mM final concentration of IPTG was added to induce the expression of fusion proteins at 25° C. overnight, and the BL21/pD441-WT bacterial broth after induction was obtained. IPTG was not added to the bacterial broth of the blank control group.

BL21/pD441-WT bacterial broth was centrifuged at 8000 rpm/min for 10 minutes. Then supernatant was discarded, and pelleted cells were collected and resuspended in buffer (50 mM KPO₄, 500 mM NaCl, 10 mM imidazole, 5% Glycerol, pH 7.0) adding with PMSF at final concentration of 0.5 mM. Then the bacteria was broken by ultrasonic breaker, with a working time of 40 min, power 200 W, ultrasonic setting of 1 s stop 2 s and alarm temperature of 15° C. After sonication, the cell crusing solution was water bathed at 80° C. for 20 minutes, during which attention was paid to regular mixing so that the cell crushing soltuion was heated evenly. Then the solution was centrifuged at 12000 rpm/min and 4° C. for 30 min, the supernatant was filtered with a 0.22 μm filter membrane, and the filtrate was collected to obtain crude cell extract.

The crude cell extract was loaded at an appropriate flow rate for Ni column affinity chromatography (the prepacked column of affinity chromatography was HisTrap FF, 5ml, 17-5255-01, GE healthcare), and the column was equilibrated with 5 CV buffer 1 after loading; then eluted with 5 CV 3% buffer 2 (50 mMKPO₄, 1 M NaCl, 5% Glycerol, pH 7.0) followed by 5 CV 50% buffer 2, and the eluate of the Ni column affinity chromatography corresponding to the peak value equal to 100 mAU or higher was collected.

The eluate corresponding to the peak value equal to 100 mAU or higher was loaded at a certain flow rate for ion exchange chromatography (the prepacked column of ion exchange was HiTrap Q HP, 5 ml, 17-1154-01, GE healthcare), and the column was equilibrated with 5 CV buffer 2 after loading, linear eluted with 0% buffer 2→60% buffer 2, and the eluate of the ion exchange chromatography corresponding to the peak value equal to 100 mAU or higher was collected.

The eluate of the ion exchange chromatography corresponding to the peak value equal to 100 mAU or higher was gel chromatographed (the prepacked column of gel chromatography was HiPrepSephacryl S-100 HR, 26 mm, 17-1194-01, GE healthcare), first the column was washed with 3CV 20% ethanol, followed by 3CV with water; after being equilibrated with 3CV 100% buffer 3 (20 mM Tris, 200 mM KCl, 0.2 mM EDTA, 10% Glycerol, pH 7.4), the sample was loaded, then the column was eluted with 1.5CV buffer 3, and the eluate was collected as purified wild-type KOD DNA polymerase fusion protein.

The purified wild-type KOD DNA polymerase fusion protein was determinated by SDS-PAGE (5% stacking gel and 12% separation gel). The protein sample was mixed with SDS-PAGE protein loading buffer (5×), and treated at 95° C. for 5 min, and then loaded. The results are shown in FIG. 2. Lane 1 shows a protein Marker (PageRuler Prestained Protein Ladder, 26616, Thermo Scientific), lane 2 shows 10 μl of 1 mg/ml purified wild-type KOD DNA polymerase fusion protein, and lane 3 shows 10 μl of 0.05 mg/ml purified wild-type KOD DNA polymerase fusion protein that was diluted 20 times. It can be seen from the results of protein electrophoresis that the size of both proteins in lane 2 and lane 3 are about 91.5 KDa, which is consistent with the molecular weight reported in the literature.

The purity of the protein obtained by gel electrophoresis was analyzed with Quantity one software. The purity of the wild-type KOD DNA polymerase fusion protein after purification can reach 95% or more.

The target protein of approximately 91.5 KDa size can not be observed in uninduced BL21/pD441-WT bacterial broth.

In the control group, the vehicle pD441-pelB was introduced into E. coli BL21 to obtain BL21/pD441-pelB. The above method was used to express and purify the protein in the control group, but the target protein of about 91.5 KDa was not obtained.

### 2. Preparation of KOD DNA Polymerase Mutant Fusion Protein

KOD DNA polymerase mutant (the amino acid sequence is sequence A) was the protein obtained by modifying amino acid residues in at least one of the positions 675, 385, 710, 674, 735, 736, 606, 709, 347, 349, 590, 676, 389, 589, 680, 384, 496 and 383 of the amino acid sequence of wild-type KOD DNA polymerase; if there is only one amino acid substitution, a single point mutant of KOD DNA polymerase is obtained, if there are two amino acid substitutions, a two-point combination mutant of KOD DAN polymerase, if there are three amino acid substitutions, a three-point combination mutant of KOD DNA polymerase is obtained, and other mutants are obtained by analogy.

A gene encoding KOD DNA polymerase mutant (the nucleotide sequence is sequence B) was the nucleotide sequence obtained by mutating at least one amino acid codon of the coding gene of wild-type KOD DNA polymerase (SEQ ID NO: 2) according to positions 675, 385, 710, 674, 735, 736, 606, 709, 347, 349, 590, 676, 389, 589, 680, 384, 496 and 383 in SEQ ID NO: 1.

KOD DNA polymerase mutants can be obtained by site-directed mutagenesis in wild-type KOD DNA polymerase, or by other existing methods.

### (1) Preparation of Recombinant Vector Expressing KOD DNA Polymerase Mutant

The recombinant vectors expressing different KOD DNA polymerase mutants were the vectors obtained by recombining different genes encoding KOD DNA polymerase mutant proteins fused with His tag into the vector pD441-pelB, and genes encoding different point mutant proteins fused with His tag were expressed using the signal peptide on the vector pD441-pelB.

The amino acid sequence of each KOD DNA polymerase mutant fusion protein was obtained by connecting 6×His tags to the C-terminus of the KOD DNA polymerase mutant shown in sequence A; wherein sequence A is the sequence obtained by mutating the amino acid sequence of wild-type KOD DNA polymerase as shown in SEQ ID NO: 1.

The nucleotide sequence of those genes encoding different KOD-type DNA polymerase mutant proteins fused with His tag were obtained by connecting codon of 6×His tag to the 3′ end of different KOD DNA polymerase mutant proteins shown in sequence B; wherein sequence B is the sequence obtained by mutating the corresponding amino acid codon on the nucleotide sequence encoding wild-type KOD DNA polymerase as shown in SEQ ID NO: 2.

Single-point mutants are taken as an example below, as shown in Table 1.

Table 1 shows the mutation position and mutation information of single-point mutant KOD DNA polymerase

| | Mutation | The original | The amino |
|---|---|---|---|
| Name of | position | amino acid | acid of |
| KOD DNA | (position | of wild-type | KOD DNA |
| polymerase | in SEQ | KOD DNA | polymerase |
| mutant | ID NO: 1) | polymerase | mutant |
| A01 | 500 | A | D/G |
| A03 | 675 | A | F/M |
| B05 | 385 | E | M/W |
| A06 | 664 | E | L/M |
| C14 | 710 | I | S/H |
| D20 | 674 | K | L/C |
| D21 | 351 | N | Y/H |
| E23 | 735 | N | H |
| F29 | 736 | Q | E/A |
| G32 | 606 | R | H/M |
| H37 | 709 | R | S/H |
| I40 | 347 | S | I/M |
| J46 | 349 | T | F/I |
| K50 | 590 | T | K/L |
| L52 | 676 | T | K/Y |
| M54 | 389 | V | I/M |
| N56 | 589 | V | H/Q |
| O60 | 680 | V | M/E |
| P63 | 384 | Y | F/W |
| Q66 | 496 | Y | L/I |
| R69 | 383 | S | T |

Multiple-point mutants are taken as an example of KOD DNA polymerase, as shown in Table 2 below.

### (2) Construction of Recombinant Bacteria

Using the same method of expression and purification as defined in step (2) of the preceding Item 1, the recombinant vector expressing different KOD type DNA polymerase mutants prepared in preceding program 1 was transducted into BL21 to obtain recombinant bacteria expressing different KOD DNA polymerase mutant fusion proteins.

### (3) Expression and Purification of Mutants

The recombinant bacteria expressing different KOD DNA polymerase mutant fusion proteins prepared in the preceding step (2) was expressed and purified using the same method as that of wild-type KOD DNA polymerase fusion protein in the preceding Item 1 to obtain different KOD DNA polymerase mutat fusion proteins.

The different KOD DNA polymerase mutant fusion proteins were determined by SDS-PAGE (the stacking gel was 5% and the separation gel was 12%), and the target proteins were obtained. The purity of those proteins in the protein gel after electrophoresis was analyzed with Quantity one software, and it was found that the purity of all the different KOD DNA polymerase mutant fusion proteins can reach 95% or more.

### Example 2

### Testing the Properties of Recombinant KOD DNA Polymerase Mutant Fusion Proteins

### 1. Detection of Polymerase Activity of Recombinant KOD DNA Polymerase Mutant Fusion Proteins

The polymerase activity was measured according to the method of Nishioka, M., et al. (2001. J. Biotechnol. 88), and one unit of enzyme activity is defined as the amount of acid-insoluble substances generated by polymerizing 10 nmol of dNTP by one unit enzyme in a 50 μl reaction system at 75° C. for 30 min.

The reaction solution was: 20 mM Tris-HCl (pH7.5), 8 mM MgCl₂, 50 μg/ml BSA, 0.15 mM each dNTPs (dATP, dTTP, dCTP and dGTP), [methyl-3H]-TTP (0.13 mCi/nmol, final concentration, (company MPBIO-FINE, product catalog number CB2272108), 150 mg/ml activated calf thymus DNA (company SIGMA-ALDRICH, product catalog number GE27-4575-01), 7.5 mM DTT, 1 μL wild-type KOD DNA polymerase (1 mg/ml), the total reaction volume was 50 μl.

The reaction solution was reacted at 75° for 30 minutes, and then the polymerase activity of wild-type KOD DNA polymerase calculated according to the definition of enzyme activity was 2U/μl.

Wild-type KOD DNA polymerase fusion proteins and KOD DNA polymerase mutant fusion proteins are taken as examples to perform the polymerization reaction as defined above.

Wherein, the KOD DNA polymerase mutants are the multiple-point mutants of KOD DNA polymerase in Table 2: AB60, AB70, AB71, CD72, CD73, EF74, EF75, EF76, GH77 and GH78;

Table 2 shows the multiple-point mutants of KOD DNA polymerase

| | Polymerase |
|---|---|
| Name | activity |
| WT (wild- | 2.0 |
| type KOD DNA | |
| polymerase) | |
| AB60 | 2.5 |
| AB70 | 3.1 |
| AB71 | 3.3 |
| CD72 | 2.8 |
| CD73 | 2.4 |
| EF74 | 2.7 |
| EF75 | 3.5 |
| EF76 | 3.2 |
| GH77 | 3.8 |
| GH78 | 4.5 |

The specific mutation positions and methods of mutants shown in Table 2 are as follows:
the amino acid sequence of AB60 is that the arginine at position 709 of SEQ ID NO: 1 is mutated to serine, and the valine at position589 of SEQ ID NO: 1 is mutated to histidine;
the amino acid sequence of AB70 is that the threonine at position 676 of SEQ ID NO: 1 is mutated to lysine, the valine at position 589 of SEQ ID NO: 1 is mutated to histidine, and the valine at position 680 SEQ ID NO: 1 is mutated to methionine;
the amino acid sequence of AB71 is that the lysine at position 674 of SEQ ID NO: 1 is mutated to leucine, the valine at position 589 of SEQ ID NO: 1 is mutated to histidine, and the valine at position 680 of SEQ ID NO: 1 is mutated to methionine;
the amino acid sequence of CD72 is that the threonine at position 676 of SEQ ID NO: 1 is mutated to lysine, the valine at position 589 of SEQ ID NO: 1 is mutated to histidine, the valine at position 680 of SEQ ID NO: 1 is mutated to methionine, and the tyrosine at position 384 of SEQ ID NO: 1 is mutated to phenylalanine;
the amino acid sequence of CD73 is that the valine at position 389 of SEQ ID NO: 1 is mutated to isoleucine, the lysine at position 674 of SEQ ID NO: 1 is mutated to leucine, the valine at position 589 of SEQ ID NO: 1 is mutated to histidine, and the valine at position 680 of SEQ ID NO: 1 is mutated to methionine;
the amino acid sequence of EF74 is that the lysine at position 674 of SEQ ID NO: 1 is mutated to leucine, the valine at position 589 of SEQ ID NO: 1 is mutated to histidine, the valine at position 680 of SEQ ID NO: 1 is mutated to methionine, the serine at position 383 of SEQ ID NO: 1 is mutated to threonine, and the tyrosine at position 384 of SEQ ID NO: 1 is mutated to phenylalanine;
the amino acid sequence of EF75 is that the threonine at position 349 of SEQ ID NO: 1 is mutated to phenylalanine, the valine at position 589 of SEQ ID NO: 1 is mutated to histidine, the threonine at position 676 of SEQ ID NO: 1 is mutated to lysine, the valine at position 680 of SEQ ID NO: 1 is mutated to methionine, and the tyrosine at position 496 of SEQ ID NO: 1 is mutated to leucine;

The amino acid sequence of EF76 is that the valine at position 589 of SEQ ID NO: 1 is mutated to histidine, the threonine at position 676 of SEQ ID NO: 1 is mutated to lysine, the valine at position 680 of SEQ ID NO: 1 is mutated to methionine, the serine at position 383 of SEQ ID NO: 1 is mutated to threonine, and the tyrosine at position 384 of SEQ ID NO: 1 is mutated to phenylalanine;

The amino acid sequence of GH77 is that the valine at position 589 of SEQ ID NO: 1 is mutated to histidine, the threonine at position 676 of SEQ ID NO: 1 is mutated to lysine, the valine at position 680 of SEQ ID NO: 1 is mutated to methionine, the serine at position 383 of SEQ ID NO: 1 is mutated to threonine, the tyrosine at position 384 of SEQ ID NO: 1 is mutated to phenylalanine, and the arginine at position 709 of SEQ ID NO: 1 is mutated to serine;

The amino acid sequence of GH78 is that the valine at position 389 of SEQ ID NO: 1 is mutated to isoleucine, the valine at position 589 of SEQ ID NO: 1 is mutated to histidine, the threonine at position 676 of SEQ ID NO: 1 is mutated to lysine, the valine at position 680 of SEQ ID NO: 1 is mutated to methionine, the serine at position 383 of SEQ ID NO: 1 is mutated to threonine, and the tyrosine at position 384 of SEQ ID NO: 1 is mutated to phenylalanine.

The KOD DNA polymerase mutant fusion proteins were prepared by adding 6×His tags to the C-terminus of each KOD DNA polymerase mutant.

The results of the activity of KOD DNA polymerase mutant fusion proteins are shown in Table 2. It can be seen that the KOD DNA polymerase mutants have polymerase activity.

The polymerase activity results of other mutants were similar.

### 2. Kinetics of Single Base Incorporation of KOD DNA Polymerase Mutant Fusion Proteins

In this example, dATP (dATP-Cy3) labeled with Cy3 fluorescent dye and substrate (P/T-2Cy5) labeled with Cy5 fluorescent dye were used to detect the relative reaction rate of recombinant KOD DNA polymerase mutants using a microplate reader, so as to approximately depict the Michaelis-Menten curve of each mutant, and the specific experimental method is as follows:

The single-stranded primers PIA (sequence 5 in the sequence listing) and P2A (sequence 6 in the sequence listing) that were both labeled with 5′ Cy5 fluorescent were mixed 1:1, then the mixture was kept at 65° C. for 10 minutes, and naturally cooled to room temperature. The obtained annealing product, i.e., the substrate P/T-2Cy5 labeled with Cy5 fluorescent dye, was stored at-20° C. in the dark.

Enzyme activity was detected by BioTek microplate reader. The reaction was carried out in 384-well plate (Corning black, clear bottom 384 plate), and the total volume of the reaction solution was 50 μl. The specific reaction system is as follows:

The reaction system is: 1U KOD polymerase mutant fusion protein, 1 μM dATP-Cy3, 10 μM dTTP, 10 μM dCTP, 10 μM dGTP and substrate P/T-2Cy5. The experiment was performed with 8 concentration gradients of 0.1, 0.2, 0.5, 1, 2, 3, 4 and 6 μM. The buffer for enzymatic reaction is 20 mM Tris-HCl1, 10 mM (NH₄)₂SO₄, 10mM KCl and 2 mM MgSO₄ with pH 8.5 @ 25° C.

Kinetic detection mode was used in the enzymatic reaction, and the data was recorded every 30 s. The parameters of measurement are:

| | |
|---|---|
| Extension | 530 nm |
| Emission | 676 nm |
| Reaction Temperature | 40° C. |
| Reaction time | 30 min |

After the reaction, the spreadsheet of data and curve of enzyme activity can be exported directly, and the reaction rate of the relative fluorescence value can be approximately calculated.

The reaction rate to the fluorescence value depends on the concentration of the substrate P/T-2Cy5, so the Km value of recombinant KOD DNA polymerase mutant, which corresponds to the concentration of the substrate when it reaches half of the maximum reaction rate, can be approximately determined by measuring the activity of the mutant under different concentrations of substrate P/T-2Cy5.

The wild-type KOD DNA polymerase fusion protein (KOD-WT) was used as a control.

The Michaelis-Menten curves of KOD DNA polymerase mutant fusion proteins were shown in FIG. 3 and Table 3. It can be seen that the polymerase activity of KOD DNA polymerase mutants is higher than that of wild type. The specific experimental results can be reffered to Table 3 below. The results of kinetic test of KOD DNA polymerase mutants showed that they had greater potential than wild type. In the sequencing process, the higher the catalytic efficiency of the enzyme reaction, the greater the value of Vmax/Km, which can speed up the reaction to a certain extent and shorten the time required for sequencing reaction. It can be seen from the experimental results that all the KOD DNA polymerase mutants have improved enzyme catalytic efficiency compared with the wild-type KOD DNA polymerase.

TABLE 3
Results of kinetic test of KOD DNA polymerase mutants

| | | Compare to WT |
|---|---|---|
| | | (Vmax/km value of |
| | | mutant to Vmax/km |
| Mutants | Vmax/km | value of wild-type) |
| AB60 | 0.95E+07 | 2.7 |
| AB70 | 1.64E+07 | 4.7 |
| AB71 | 1.68E+07 | 4.2 |
| CD72 | 1.06E+07 | 3.9 |
| CD73 | 2.26E+07 | 6.5 |
| EF74 | 1.70E+07 | 6.2 |
| EF75 | 2.58E+07 | 7.5 |
| EF76 | 6.37E+06 | 2.5 |
| GH77 | 1.32E+07 | 3.4 |
| GH78 | 2.01E+07 | 7.3 |

### INDUSTRIAL APPLICATION

The experiment of the present invention proves that aiming at further extending the read length of sequencing, shortening the reaction time and other improvement goals, the present invention further modified the KOD DNA polymerase based on the previous work, while protecting original functional sites involved in this area of the DNA polymerase B family to ensure they are still capable to complete the original basic functions. The recombinant KOD polymerase has higher reaction rate of DNA polymerase in sequencing and increased reaction read length. Compared with the wild-type DNA polymerase, the recombinant DNA polymerase exhibits better reaction rate, better affinity and other advantages in aspect of catalysis.

What is claimed is:

1. A protein,
wherein the sequence of the protein has at least 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% identity with the wild-type KOD DNA polymerase, and has the following substitutions in the corresponding wild-type KOD DNA polymerase amino acid sequence:
the threonine at position 676 with lysine; the valine at position 589 with histidine; and
the valine at position 680 with methionine;
wherein the protein has DNA polymerase activity;
wherein the amino acid sequence of wild-type KOD DNA polymerase is shown in SEQ ID NO: 1.

2. The protein according to claim 1, wherein the sequence of the protein has substitutions at least at one of the following positions in the corresponding wild-type KOD DNA polymerase amino acid sequence: 383, 384, 389, 349, 496, and/or 709, and the protein has DNA polymerase activity.

3. The protein according to claim 1, wherein the sequence of the protein comprises the tag sequence in the terminal of the protein, and the protein has DNA polymerase activity.

4. The protein according to claim 2, wherein the sequence of the protein comprises the tag sequence in the terminal of the protein, and the protein has DNA polymerase activity.

5. The protein according to claim 1, wherein the sequence of the protein has at least one of the following substitutions in the corresponding wild-type KOD DNA polymerase amino acid sequence:
the serine at position 383 with threonine;
the tyrosine at position 384 with phenylalanine or tryptophan;
the valine at position 389 with isoleucine or methionine;
the threonine at position 349 with phenylalanine or isoleucine;
the tyrosine at position 496 with leucine or isoleucine;
the arginine at position 709 with serine or histidine.

6. The protein according to claim 4, wherein the sequence of the protein has the following substitutions in the corresponding wild-type KOD DNA polymerase amino acid sequence: the threonine at position 676 with lysine, the valine at position 589 with histidine, the valine at position 680 with methionine, and the tyrosine at position 384 with phenylalanine;
and the protein has DNA polymerase activity.

7. The protein according to claim 4, wherein the sequence of the protein has the following substitutions in the corresponding wild-type KOD DNA polymerase amino acid sequence: the threonine at position 676 with lysine, the valine at position 589 with histidine, the valine at position 680 with methionine, the threonine at position 349 with phenylalanine, and the tyrosine at position 496 with leucine;
and the protein has DNA polymerase activity.

8. The protein according to claim 4, wherein the sequence of the protein has the following substitutions in the corresponding wild-type KOD DNA polymerase amino acid sequence: the threonine at position 676 with lysine, the valine at position 589 with histidine, the valine at position 680 with methionine, the serine at position 383 with threonine, and the tyrosine at position 384 with phenylalanine;
and the protein has DNA polymerase activity.

9. The protein according to claim 4, wherein the sequence of the protein has the following substitutions in the corresponding wild-type KOD DNA polymerase amino acid sequence: the threonine at position 676 with lysine, the valine at position 589 with histidine, the valine at position 680 with methionine, the serine at position 383 with threonine, the tyrosine at position 384 with phenylalanine, and the arginine at position 709 with serine;
and the protein has DNA polymerase activity.

10. The protein according to claim 4, wherein the sequence of the protein has the following substitutions in the corresponding wild-type KOD DNA polymerase amino acid sequence: the threonine at position 676 with lysine, the valine at position 589 with histidine, the valine at position 680 with methionine, the serine at position 383 with threonine, the tyrosine at position 384 with phenylalanine, and the valine at position 389 with isoleucine;
and the protein has DNA polymerase activity.

11. A DNA molecule encoding the protein of claim 1.

12. A recombinant expression vector comprising the DNA molecule of claim 10.

13. A transformant, wherein the transformant is obtained by transducting the recombinant expression vector of claim 10 into host cell;
preferably the host cell is bacteria cell;
more preferably, the bacteria cell is BL21.

14. A method for preparing a DNA polymerase, comprising culturing the transformant of claim 13 to express the DNA polymerase;
preferably the method further comprising purifying the DNA polymerase.

15. A kit, wherein the kit comprising the protein of claim 1;
preferably the kit further comprising the specification.

16. A method for amplifying DNA, wherein the method comprising using the protein of claim 1.

17. A method for amplifying DNA, wherein the method comprising using the kit of claim 15.


SEQUENCE LISTING (6 sequences)

SEQ ID NO: 1 - protein, 774 aa, synthetic construct
  REGION 1..774: note = Amino acid sequence of wild-type KOD DNA polymerase
MILDTDYITEDGKPVIRIFKKENGEFKIEYDRTFEPYFYALLKDDSAIEEVKKITAERHGTVVTVKRVEKVQKKFLGRPVEVWKLYFTHPQDVPAIRDKIREHPAVIDIYEYDIPFAKRYLIDKGLVPMEGDEELKMLAFAIATLYHEGEEFAEGPILMISYADEEGARVITWKNVDLPYVDVVSTEREMIKRFLRVVKEKDPDVLITYNGDNFDFAYLKKRCEKLGINFALGRDGSEPKIQRMGDRFAVEVKGRIHFDLYPVIRRTINLPTYTLEAVYEAVFGQPKEKVYAEEITTAWETGENLERVARYSMEDAKVTYELGKEFLPMEAQLSRLIGQSLWDVSRSSTGNLVEWFLLRKAYERNELAPNKPDEKELARRRQSYEGGYVKEPERGLWENIVYLDFRSIAPSIIITHNVSPDTLNREGCKEYDVAPQVGHRFCKDFPGFIPSLLGDLLEERQKIKKKMKATIDPIERKLLDYRQREIKILANSYYGYYGYARARWYCKECAESVTAWGREYITMTIKEIEEKYGFKVIYSDTDGFFATIPGADAETVKKKAMEFLKYINAKLPGALELEYEGFYKRGFFVTKKKYAVIDEEGKITTRGLEIVRRDWSEIAKETQARVLEALLKDGDVEKAVRIVKEVTEKLSKYEVPPEKLVIHEQITRDLKDYKATGPHVAVAKRLAARGVKIRPGTVISYIVLKGSGRIGDRAIPFDEFDPTKHKYDAEYYIENQVLPAVERILRAFGYRKEDLRYQKTRQVGLSAWLKPKGT

SEQ ID NO: 2 - DNA, 2325 bp, synthetic construct
  misc_feature 1..2325: note = Nucleotide sequence of wild-type KOD-type DNA polymerase
  source 1..2325: mol_type = other DNA
atgattctggacaccgattacatcaccgaagatggcaagccagttatccgcattttcaaaaaagagaatggtgaattcaagatcgaatatgatcgtaccttcgagccgtacttctatgctctgctgaaagacgatagcgcgattgaggaggtcaagaaaatcaccgcggagcgtcacggtacggttgttaccgtgaaacgcgtggagaaagtccagaagaaatttctgggtcgcccggttgaagtgtggaagctgtactttacgcatccgcaagatgttccggcgattcgcgataagattcgtgagcacccggcagtcattgacatctacgagtatgacattccgttcgccaagcgttatctgatcgataagggtctggtcccgatggagggtgacgaagaactgaagatgctggcgttcgccatcgccactctgtaccacgagggtgaagagtttgccgagggtccgatcttgatgatttcctacgcggacgaagagggcgcacgtgttatcacgtggaaaaatgttgatctgccgtatgttgacgtcgtaagcaccgagcgtgagatgatcaaacgttttctgcgcgttgttaaagaaaaagatcctgacgtgctgatcacctacaacggtgacaatttcgatttcgcgtacctgaagaaacgttgcgaaaaactgggtattaacttcgcgctgggtcgcgatggctctgaaccgaagatccagcgcatgggtgatcgttttgcggtcgaggtgaagggtcgcattcatttcgacctgtacccggtgattcgtcgtaccatcaacttgccgacttacaccctggaagccgtctatgaagctgtatttggtcaaccgaaagaaaaagtgtacgctgaggaaattacgacggcgtgggaaaccggtgagaacctggagcgcgttgcacgttattctatggaggacgcgaaagttacctacgaactgggtaaagagttcctgccgatggaggcccaactgtcccgtctgatcggccaaagcctgtgggacgttagccgcagcagcaccggtaacttagttgaatggttcttgctgcgtaaggcatacgaacgcaatgagctggcgccgaacaaaccggacgagaaagaattggcgcgtcgccgccagagctatgagggtggttatgtcaaagaaccggagcgcggcttgtgggagaacatcgtctatttggattttcgtagcattgcaccgagcatcattatcacgcataatgtgagcccggatacgttgaatcgtgagggctgtaaggaatacgacgtggcgcctcaggttggccaccgtttctgcaaggactttccgggctttatcccgagcctgctgggtgatttgctggaggaacgtcagaaaatcaagaagaagatgaaagcaaccattgatccgatcgagcgcaaattactggactaccgtcaacgtgaaatcaagatcctggcgaattcgtattatggttactatggctacgcgcgtgcgcgctggtattgcaaagagtgtgccgagagcgtgaccgcttggggtcgtgagtacattaccatgacgatcaaagagattgaagagaaatacggctttaaggttatctatagcgacaccgacggtttctttgcaactatccctggcgcagacgcagaaaccgttaagaaaaaggcaatggagtttctgaagtatatcaacgcgaagttgccaggcgccctggaactggagtacgagggcttctacaagcgtggctttttcgtgacgaaaaagaaatacgctgttattgatgaagagggcaagatcacgacccgtggcctggaaattgtgcgccgtgattggagcgaaattgcaaaagaaacgcaagcgcgtgtgctggaagcgctgctgaaggacggcgacgtcgaaaaagctgtgcgtattgttaaagaggtcaccgagaagctgagcaaatacgaggtcccgccagagaaattggtgattcacgaacagattacgcgtgacctgaaagactataaggccaccggtccgcatgtcgcagtggcgaagcgcctggcggctcgcggtgtgaagatccgtccgggtaccgtcattagctatatcgtgctgaagggcagcggtcgtatcggcgaccgtgcgattccgttcgacgaatttgatccgaccaaacacaaatatgatgcggaatactatattgagaaccaagtgctgccagccgttgagcgtattctgcgcgccttcggttaccgcaaggaagatctgcgttaccagaaaactcgtcaggtcggtctgtccgcatggctgaaaccgaagggcacctga

SEQ ID NO: 3 - DNA, 25 bp, synthetic construct
  misc_feature 1..25: note = Primer Cloning-F
  source 1..25: mol_type = other DNA
ggtttttttatggggggagtttagg

SEQ ID NO: 4 - DNA, 25 bp, synthetic construct
  misc_feature 1..25: note = Primer Cloning-R
  source 1..25: mol_type = other DNA
catctcatctgtaacatcattggca

SEQ ID NO: 5 - DNA, 37 bp, synthetic construct
  misc_feature 1..37: note = Primer P1A
  source 1..37: mol_type = other DNA
cgtgtatgcgtaataggatcccgactcactatggacg

SEQ ID NO: 6 - DNA, 37 bp, synthetic construct
  misc_feature 1..37: note = Primer P2A
  source 1..37: mol_type = other DNA
cgtgtatcgtccatagtgagtcgggatcctattacgc